(19) United States
(12) Patent Application Publication
(10) Pub. No.: US 2008/0311106 A1
(43) Pub. Date: Dec. 18, 2008

(54) Product Comprising a C4bp Core Protein and a monomeric Antigen, and Its Use

(75) Inventor: Fergal Hill, Saint-Beauzire (FR)

Correspondence Address: NIXON & VANDERHYE, PC, 901 NORTH GLEBE ROAD, 11TH FLOOR, ARLINGTON, VA, 22203, US

(73) Assignee: IMAXIO, LYON (FR)

(21) Appl. No.: 10/567,928

(22) PCT Filed: Aug. 12, 2004
(86) PCT No.: PCT/IB2004/002717

§ 371 Date: Jul. 30, 2008

(30) Foreign Application Priority Data
Aug. 12, 2003 (EP) PCTEP0308926

(51) Int. Cl. A61K 39/395; A61K 39/00; A61K 39/015; C12N 15/63; C12N 5/06; A61P 31/12; C12N 1/21; A61K 39/145; C12P 21/00

(52) U.S. Cl. 424/130.1; 424/184.1; 424/185.1; 424/272.1; 424/209.1; 800/6; 435/320.1; 435/252.3; 435/325

(57) ABSTRACT

The invention provides a product which comprises a C4bp core protein and a monomeric antigen, desirably in the form of a fusion protein. Monomeric antigens include malarial and influenza antigens. The C4bp core protein provides for assembly of multimeric complexes of the monomeric antigen, or mixtures thereof. The complexes are useful as vaccines.

This application claims the benefit of priority of PCT/EP2003/008926, the contents of which are incorporated herein by reference.

### FIELD OF THE INVENTION

This invention relates to macromolecular assemblies, such as fusion proteins, comprising an adjuvant and an antigen, which assemblies provoke an enhanced immune response to the antigen in comparison to the antigen alone.

### BACKGROUND OF THE INVENTION

Adjuvants enhance the immune response to antigens and are therefore useful in vaccines. However, there are only a limited number of adjuvants approved for use in humans, and as stronger adjuvants are known from research on animals, a clear need exists for stronger immunological adjuvants which are safe to use in man. For a recent review, see “Advances in vaccine adjuvants” (Nature Biotechnology, 1999, Volume 17, pages 1075-1081) and “Recent advances in the discovery and delivery of vaccine adjuvants” (Nature Reviews in Drug Discovery, 2003, Volume 2, pages 727-735).

The complement system consists of a set of serum proteins that are important in the response of the immune system to foreign antigens. The complement system becomes activated when its primary components are cleaved and the products, alone or with other proteins, activate additional complement proteins resulting in a proteolytic cascade. Activation of the complement system leads to a variety of responses including increased vascular permeability, chemotaxis of phagocytic cells, activation of inflammatory cells, opsonisation of foreign particles, direct killing of cells and tissue damage.

Activation of the complement system may be triggered by antigen-antibody complexes (the classical pathway) or a normal slow activation may be amplified in the presence of cell walls of invading organisms such as bacteria and viruses (the alternative pathway). The complement system interacts with the cellular immune system through a specific pathway involving C3, a protein central to both classical and alternative pathways. The proteolytic activation of C3 gives rise to a large fragment (C3b) and exposes a chemically reactive internal thiolester linkage which can react covalently with external nucleophiles such as the cell surface proteins of invading organisms or foreign cells. As a result, the potential antigen is “tagged” with C3b and remains attached to that protein as it undergoes further proteolysis to iC3b and C3d,g. The latter fragments are, respectively, ligands for the complement receptors CR3 and CR2; (CR2 is also referred to as CD21). Thus the labelling of antigen by C3b can result in a targeting mechanism for cells of the immune system bearing these receptors.

That such targeting is important for augmentation of the immune response is first shown by experiments in which mice were depleted of circulating C3 and then challenged with an antigen (sheep erythrocytes). Removal of C3 reduced the antibody response to this antigen (M. B. Pepys, J. Exp. Med., 140, 126-145, 1974). The role of C3 was confirmed by studies in animals genetically deficient in either C3 or the upstream components of the complement cascade which generate C3b, i.e. C2 and C4 (J. M. Ahearn and D. T. Fearon, Adv. Immunol., 46, 183-219, 1989). More recently, it has been shown that linear conjugation of a model antigen with more than two copies of the murine C3d fragment sequence resulted in a very large (1000-10000-fold) increase in antibody response in mice compared with unmodified antigen controls (P. W. Dempsey et al, Science, 271, 348-350, 1996; WO96/17625, PCT/GB95/02851). The increase could be produced without the use of conventional adjuvants such as Freund's complete adjuvant, which is too toxic to be used in humans. The mechanism of this remarkable effect was demonstrated to be high-affinity binding of the multivalent C3d construct to CR2 on B-cells, followed by co-ligation of CR2 with another B-cell membrane protein, CD19 and with membrane-bound immunoglobulin to generate a signal to the B-cell nucleus.

However, it has proved difficult to produce large amounts of homogenous recombinant proteins containing three copies of C3d. The principal problems have been:

i) the genetic instability of the constructs containing (three) repeated sequences; and
ii) the folding (or solubilisation and refolding) of the recombinant protein from inclusion bodies formed in Escherichia coli.

One approach taken to minimise the genetic instability of constructs containing repeated copies of the C3d gene is described in WO99/35260 and WO01/77324. The technology described in these applications is to use non-identical sequences of DNA encoding repeats of C3d.

A multimerisation system using the complement 4 binding protein (C4bp) is described in WO 91/11461. Human C4b-binding protein (C4BP) is a plasma glycoprotein of high molecular mass (570 kDa) which has a spider like structure made of seven identical alpha-chains and a single beta-chain. The C4bp alpha chain has a C-terminal core region responsible for assembly of the molecule into a multimer. According to the standard model, the cysteine at position +498 of one C4bp monomer forms a disulphide bond with the cysteine at position +510 of another monomer. A minor form comprising only seven alpha-chains has also been found in human plasma. The natural function of this plasma glycoprotein is to inhibit the classical pathway of complement activation.

Most of the alpha-chain of C4bp is composed of eight tandemly arranged domains of approximately 60 amino acids in length known as complement control protein (CCP) repeats. WO91/11461 proposes that the ability of the C4bp protein to multimerise can be used to make fusion proteins comprising all or part of C4bp and a biological protein of interest. Inclusion of one or more of the CCP repeats (also known as SCRs) were preferred in the fusion proteins described in WO 91/11461.

WO91/11461 suggests that fusion proteins may be used as vaccines. A number of specific proteins comprising at least one C4bp SCR region fused to a fragment of hepatitis B e antigen were made. The e antigen fragments used are core antigen fragments which are capable of forming multimer structures.

Libyh M. T. et al., (1997, Blood, 90, 3978-3983) demonstrate that all CCPs can be deleted (leaving only the C-terminal 57 amino acids) without preventing multimerisation. This C-terminal region of C4bp is referred to herein as the C4bp core.

Self-assembling multimeric soluble CD4-C4bp fusion protein have also been demonstrated in Shinya et al (1999, Biomed & Pharmacother, Vol. 53: 471) where the fusion proteins were expressed in the human 293 cell line.

The use of C4bp is also described in Oudin et al. (2000, Journal of Immunology, Vol. 164:1505).

Christiansen et al. (2000, Journal of Virology, Vol. 74:4672) discuss the therapeutic use of a CD46-C4bp fusion protein.

WO2004/020639 provides a method for obtaining a recombinant fusion protein in a prokaryotic host, comprising a scaffold of a C-terminal core protein of C4bp alpha chain optionally fused to a heterologous polypeptide, said recombinant fusion protein being capable of forming multimers in soluble form in a prokaryotic host cell.

### SUMMARY OF THE INVENTION

The present invention is based on a novel finding in relation to a particular class of C4bp fusion proteins. Some of the prior art discussed above proposes the use of C4bp fusion proteins to deliver a therapeutic protein of interest, on the basis that the C4bp moiety is essentially an inert carrier.

WO91/11461 discussed above, exemplifies fusion proteins which comprise a multimer-forming antigen.

In contrast, the present invention is based on the appreciation that antigens which naturally form multimers are not desirable for fusions to the C4bp core, as such antigens may in fact interfere with assembly of the C4bp core into multimers. In addition, it has been demonstrated that surprisingly, fusions of a C4bp core to a monomeric antigen provoke a strong immune response to the antigen. In the accompanying examples, it is shown that a fusion protein comprising a monomeric antigen provoked a high titre, inhibitory antibody response compared to a lower titre, non-inhibitory antibody response when antigen was injected with Freund's adjuvant.

Thus the present invention provides a method for increasing the immunogenicity of a monomeric antigen by combining it in a complex with a C4bp core protein. In a preferred method, the monomeric antigen is covalently bound to a C4bp core protein. In a highly preferred method, the monomeric antigen is genetically fused to a C4bp core protein.

The invention also provides a method for inducing high titres of antibodies against an antigen, and a use for the high titre antisera produced through the use of the method in the prevention and/or in the treatment of infectious and malignant diseases by passive immunisation. In a preferred method, the high titre antibodies against the antigen are partly purified by isolating the immunoglobulin fraction of the hyperimmune plasma or serum, and in a highly preferred method, the immunoglobulin fraction of the hyperimmune plasma or serum is isolated from individuals of the same species in which the antisera will be used to prevent or treat infectious or malignant diseases.

The present invention thus provides a product which comprises:
a C4bp core protein; and
a monomeric antigen.

The first and second components may be in the form of a fusion protein. Alternatively, they may be coupled chemically, through an amino acid side chain either of the first component or through the side chain of an amino acid which has been added to the first component specifically to enable the chemical coupling of the second component.

The first and second components may be tightly but noncovalently bound. For example, the side chain of an amino acid of the first component may be modified to have an additional biotin group, and this biotin can be used to combine with streptavidin (where streptavidin is the second component) or an antigen fused to streptavidin can be combined with the first component through this biotin. In another possibility, biotinylated antigen and biotinylated first component can be held together firmly but noncovalently by adding streptavidin and purifying the complexes which result.

For the avoidance of doubt, the designation of “first” and “second” components does not imply or indicate a specific linear order in the product of the two components. The two components may be joined in any order.

Thus where both components are polypeptides and the product is made as a fusion protein, the N- to C-terminal order of the two components may be in any permutation.

The invention further provides nucleic acid encoding a fusion protein of said first and second components. The invention also provides vectors comprising said nucleic acids and host cells carrying said vectors.

In another embodiment, the invention provides a method of making a product comprising:
a C4bp core protein; and
a polypeptide monomeric antigen,
the method comprising expressing nucleic acid encoding the two components in the form of a fusion protein, and recovering the product.

In another embodiment, the invention provides a method of making a product comprising:
a C4bp core protein;
a non-polypeptide monomeric antigen,
the method comprising expressing nucleic acid encoding the C4bp core protein, joining said core protein to the antigen, and recovering the product.

The methods of making the product may be performed in eukaryotic or prokaryotic cells.

The invention also provides a method of inducing an immune response to an antigen which method comprises administering to a subject an effective amount of a product according to the invention.

The invention also provides the use of a product of the invention for a method of treatment of the human or animal body, particularly a method of inducing an immune response.

The invention further provides a pharmaceutical composition comprising a product of the invention in association with a pharmaceutically acceptable carrier or diluent.

The invention further provides a method of preparing a protective immune serum for use in passive immunization against an infectious agent, said method comprising vaccinating an animal with a product of the invention, recovering antiserum from said animal. The antiserum may then be used in a method of passive immunization of a human subject. The human subject may be a subject with, or at risk from, infection with the infectious agent.

### DESCRIPTION OF THE DRAWINGS

FIG. 1 shows an alignment of C4bp core proteins.

FIG. 2 shows the results of expression of proteins in E. coli.

FIG. 3 shows a comparison of a protein of the invention run on gels under reducing and non-reducing conditions.

### DETAILED DESCRIPTION OF THE INVENTION

### Core Protein of C4bp Alpha Chain.

This is referred to herein as the “C4bp core protein” or “core protein”, or “C4bp scaffold”. The terms are used interchangeably. This protein may be a mammalian C4bp core protein or a fragment thereof capable of forming multimers and capable of acting as an adjuvant, or a synthetic or chimeric variant thereof capable of forming multimers and capable of acting as an adjuvant.

In this invention, a C4bp core protein or a fragment of the C4bp alpha chain comprising the core protein, described in further detail herein, serves as an adjuvant. The human C4bp core protein of SEQ ID NO: 1 corresponds to amino acids +493 to +549 of full length C4bp protein sequence known in the art to form multimers.

The invention moreover comprises the use of derivatives of the C4bp core to increase the immunogenicity of antigens. Such derivatives comprise mutants thereof, which may contain amino acid deletions, additions especially the addition of cysteine residues or substitutions, hybrids or chimeric molecules formed by fusion of parts of different members of the C4bp families and/or circular permutated protein scaffolds, subject to the maintenance of the adjuvant property described herein.

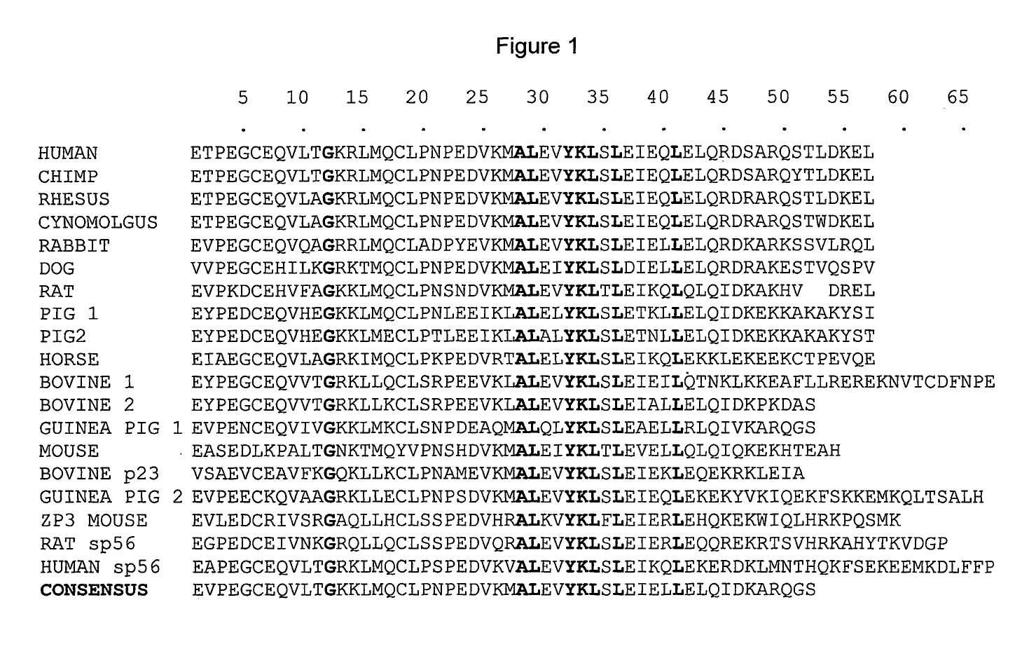

The invention may also use artificial consensus C4bp sequences based on the alignment of the C4bp core sequences from multiple species. One example of this class of chimeric molecule, of the very many possible, is given below (SEQ ID:20, FIG. 1).

Thus the adjuvant may be a C4bp core and optionally one or more SCRs fused to the core.

In a particularly preferred embodiment, the C4bp component of the product of the invention is the core protein of C4bp alpha chain, i.e. the core protein as defined herein not linked to any C4bp SCR sequences. In such an embodiment, the C4bp core desirably consists of the residues 1-57 of SEQ ID NO:1 or the corresponding residues of homologue thereof, or a fragment of at least 47 amino acids of SEQ ID NO:1 or homologue thereof.

The C4bp core of a product of the invention may additionally comprise N- or C-terminal extensions such as flexible linkers. Generally such linkers are a few amino acids in length, such as from 1 to 20, e.g. from 2 to 10 amino acids in length. One such linker is a (Glyₘ-Ser)ₙ linker, where m and n are each independently from 1 to 4. These are used in the art to attach protein domains to each other. Thus the first component may be linked to the second by such a linker.

It is preferred that when the first component is the C4bp core, it is at the C-terminal of the product.

The sequences of a number of mammalian C4bp proteins are available in the art. These include human C4bp core protein (SEQ ID NO: 1). There are a number of homologues of human C4bp core protein available in the art. There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene, chromosome or genome duplication, i.e. the common ancestor of the genes occurred since the last speciation event.

For example, a search of GenBank and raw genomic trace and EST (expressed sequence tag) databases indicates mammalian C4bp core SEQ ID NO:1 homologue proteins in species including chimpanzees, rhesus monkeys, rabbits, rats, dogs, horses, mice, guinea pigs, pigs and cattle. Paralogues and orthologues of the C4bp of SEQ ID NO:1 have been included in the alignment in FIG. 1.

In total, an alignment of SEQ ID NOs: 1-19 is shown as FIG. 1. It can be seen that all nineteen sequences have a high degree of similarity, though with a greater degree of variation at the C-terminal end. Further C4bp core proteins may be identified by searching databases of DNA or protein sequences, using commonly available search programs such as BLAST.

Where a C4bp protein from a desired mammalian source is not available in a database, it may be obtained using routine cloning methodology well established in the art. In essence, such techniques comprise using nucleic acid encoding one of the available C4bp core proteins as a probe to recover and to determine the sequence of the C4bp core proteins from other species of interest. A wide variety of techniques are available for this, for example PCR amplification and cloning of the gene using a suitable source of genomic DNA or mRNA (e.g. from an embryo or an actively dividing differentiated or tumour cell), or by methods comprising obtaining a cDNA library from the mammal, e.g. a cDNA library from one of the above-mentioned sources, probing said library with a known C4bp nucleic acid under conditions of medium to high stringency (for example 0.03M sodium chloride and 0.03M sodium citrate at from about 50° C. to about 60° C.), and recovering a cDNA encoding all or part of the C4bp protein of that mammal. Where a partial cDNA is obtained, the full length coding sequence may be determined by primer extension techniques.

A fragment of a C4bp core protein capable of forming multimers may comprise at least 47 amino acids, preferably at least 50 amino acids. The ability of the fragment to form multimers may be tested by expressing the fragment in a prokaryotic host cell according to the invention, and recovering the C4bp fragment under conditions which result in multimerisation of the full 57 amino acid C4bp core, and determining whether the fragment also forms multimers. Desirably a fragment of C4bp core comprises at least residues 6-52 of SEQ ID NO: 1 or the corresponding residues of its homologues.

Variants of C4bp core and fragments capable of forming multimers, which variants likewise retain the ability to form multimers (which may be determined as described above for fragments) may also be used. The variant will preferably have at least 70%, more preferably at least 80%, even more preferably at least 90%, for example at least 95% or most preferably at least 98% sequence identity to a wild type mammalian C4bp core or a multimer-forming fragment thereof.

In one aspect, the C4bp core will be a core which includes the glycine appears at position 12, the alanine which appears at position 28, the leucines which appear at positions 29, 34, 36, and 41 and the tyrosine which appears at position 32 and the lysine which appears at position 33 and preferably the two cysteine residues which appear at positions 6 and 18 of SEQ ID No: 1. Desirably, the variant will retain the relative spacing between these residues.

The above-specified degree of identity will be to any one of SEQ ID NOs: 1-20 or a multimer-forming fragment thereof.

Most preferably the specified degree of identity will be to SEQ ID NO:1 or a multimer-forming fragment thereof.

The degree of sequence identity may be determined by the algorithm GAP, part of the “Wisconsin package” of algorithms widely used in the art and available from Accelrys (formerly Genetics Computer Group, Madison, Wis.). GAP uses the Needleman and Wunsch algorithm to align two complete sequences in a way that maximises the number of matches and minimises the number of gaps. GAP is useful for alignment of short closely related sequences of similar length, and thus is suitable for determining if a sequence meets the identity levels mentioned above. GAP may be used with default parameters.

Synthetic variants of a mammalian C4bp core protein include those with one or more amino acid substitutions, deletions or insertions or additions to the C- or N-termini. Substitutions are particularly envisaged. Substitutions include conservative substitutions. Examples of conservative substitutions include those respecting the groups of similar amino acids often called the Dayhoff groups. These are as follows:

| | |
|---|---|
| Group 1 | D, E, N, Q |
| Group 2 | I, L, V, M |
| Group 3 | F, Y, W |
| Group 4 | K, R, H |
| Group 5 | S, P, T, A, G |
| Group 6 | C |

Examples of fragments and variants of the C4bp core protein which may be made and tested for their ability to form multimers and to act as an adjuvant thus include SEQ ID NOs: 37 to 44, shown in Table 1 below:

TABLE 1

| | | |
|---|---|---|
| A | B | C |

| | | |
|---|---|---|
| 37 | ----GCEQVLTGKRLMQCLPNPEDVKMALEVYKLSLEIEQLELQRDSARQS------ | 100 |
| 38 | ETPEGCEQVLTGKRLMQCLPNPEDVKMALEVYKLSLEIEQLELQRDSARQS------ | 100 |
| 39 | ----GSEQVLTGKRLMQSLPNPEDVKMALEVYKLSLEIEQLELQRDSARQSTLDKEL | 96 |
| 40 | ETPEGCEQVLTGKRLMQCLPNPEDVKMALEIYKLTLEIEQLELQRDSARQSTLDKEL | 96 |
| 41 | ETPEGCEQVLTGKRLMQCLPNPEDVKMALEIYKLSLEIKQLELQRDSARQSTLDKEL | 96 |
| 42 | ---EGCEQILTGKRLMQCLPDPEDVKMALEIYKLSLEIKQLELQRDRARQSTL---- | 91 |
| 43 | ETPEGCEQVLTGKRLMQCLPNPEDVKMALEVYKLSLEIKQLELQRDRARQSTLDKEL | 96 |
| 44 | ---EGCEQILTGKRLMQCLPNPEDVKMALEIYKLSLEIEQLELQRDRARQSTLDK-- | 95 |

A = SEQ ID NO; B = sequence, C = % identity, calculated by reference to a fragment of SEQ ID NO:1 of the same length.

Where deletions of the sequence are made, apart from N- or C-terminal truncations, these will preferably be limited to no more than one, two or three deletions which may be contiguous or non-contiguous.

Where insertions are made, or N- or C-terminal extensions to the core protein sequence, these will also be desirably limited in number so that the size of the core protein does not exceed the length of the wild type sequence by more than 20, preferably by no more than 15, more preferably by no more than 10, amino acids. Thus in the case of SEQ ID NO: 1, the core protein, when modified by insertion or elongation, will desirably be no more than 77 amino acids in length.

### Antigen.

An antigen is any molecule capable of being recognized by an antibody or T-cell receptor. However, not all antigens are immunogens. An immunogen is any substance which elicits an immune response. In one aspect, the present invention enables antigens which are not immunogens to become immunogens, and those antigens which are weak immunogens to become better immunogens.

An important characteristic of the present invention is that monomeric antigens are highly preferred when antigens are produced by being genetically fused to the C4bp core because they do not impede the assembly of the C4bp core protein into an oligomeric and therefore functional form.

However in an alternative aspect, the antigens may be non-monomeric when they are coupled chemically or non-covalently to the C4bp core protein.

A monomeric antigen may thus fall into two main groups:

1) An antigen which is a fragment or variant of a parent protein which in its natural state is multimeric (i.e. dimeric or a higher order multimer), but which antigen itself does not form multimers under conditions in which the parent protein does form such multimers; and
2) An antigen which in its natural state is a monomer.

Examples of both types of antigen are discussed further herein below.

Monomeric antigens have in common that they can be encoded on a single piece of DNA and when this DNA is fused to DNA encoding a C4bp core protein and subsequently translated into protein, the antigen is linked through a unique point on the antigen to a single C4bp core protein chain. A simple example of such an antigen would be lysozyme from hen egg white. The cDNA encoding the full-length lysozyme open reading frame can be fused to the C4bp core open reading frame in such a manner that the assembly of the C4bp part of the resulting fusion protein is not impeded.

After biosynthesis, a single polypeptide chain fused to a C4bp core may be processed, for example by proteases, thus generating new N- and C-termini within the polypeptide chain. If the two or more chains generated by proteolytic cleavage remain attached to one another through, for example, disulphide bonds, the C4bp fusion protein will, at the end of processing have attached to it a protein which would normally not be considered monomeric. However, for the purposes of this invention, proteins of this type are considered monomeric because they are encoded as a single fusion protein in a single open reading frame. An example of this type would be proinsulin, which is processed after biosynthesis to have two chains, called A and B, which are linked by disulphide bonds. A fragment of proinsulin, called the C peptide, is removed following proteolytic processing of the precursor fusion protein.

The monomeric antigen may be derived from a protein which is not necessarily monomeric in its natural state. Thus many antigens found in a polymeric state in Nature can be modified, for example by protein engineering techniques, so that they become monomeric. There are three examples. As example of such an antigen is one derived from the influenza virus hemagglutinin protein. This is well known to form a complex trimeric structure in its natural state (Wilson et al. Nature 289, 366-373, 1981). However, it is possible, by removing the coiled coil responsible for trimerizing the molecule to obtain a monomeric fragment. A specific example is provided by the work of Jeon and Arnon (Viral Immunology 15, 165-176, 2002). These authors used only residues 96-261 of the hemagglutinin in order to have a fragment encompassing only the globular region of the hemagglutinin.

Another example is the Plasmodium merozoite surface protein 1 (MSP1). This large (approximately 200 kDa) protein decorates the surface of merozoites which are responsible for the blood stage of malaria infections. It is normally fixed to the surface of merozoites through a C-terminal GPI anchor (where GPI is glycosylphosphatidylinisotol). This GPI anchor is preceded by a hydrophobic stretch of amino acids. As a consequence of this anchor, neither the full-length MSP1, nor the C-terminal fragment called MSP1.19 (which remains membrane-bound even as the merozoite invades erythrocytes) is ever found in a monomeric state in nature. The same applies to many membrane proteins which have a single hydrophobic transmembrane region. The present invention is best practised by deleting these hydrophobic stretches. See the example below which describes the fusion of MSP1.19 proteins to C4bp core proteins.

Thus in one preferred aspect of the invention, the product of the invention is a fusion of a plasmodium MSP1 monomeric antigenic fragment fused to a C4bp core protein. The plasmodium MSP1 antigenic fragment may comprise from about 50 to about 200, preferably from about 50 to about 150, amino acids. The antigenic fragment may be from any plasmodium species, such as Plasmodium falciparum or Plasmodium vivax or Plasmodium ovale or Plasmodium malariae (all of which are capable of causing illness in humans) or Plasmodium yoelii.

Although deletions are the easiest method of rendering monomeric otherwise oligomeric proteins, in some cases, mutating one or more amino acids may suffice. An example of this is the Cpn10 protein, which in its natural state is a heptameric protein, like the C4bp core in its principal isoforms. The mutation of a single amino acid in Cpn10 converts it into a monomeric mutant which makes it suitable for fusion to a C4bp core protein (Guidry et al. BMC Biochemistry 4, 14-26, 2003). An alternative approach to monomerize this protein was to delete N-terminal or C-terminal amino acids (Llorca et al. Biochem. Biophysica Acta 1337, 47-56, 1997; Seale and Horowitz, J. Biol. Chem. 270, 30268-30270, 1995) and thereby the regions responsible for inter-subunit interaction.

In general, for protein that will have a strong propensity to assemble into oligomeric structures (such as viral capsid proteins) thus disrupting the assembly of a C4bp core protein to which it is fused, the principles of deleting the regions responsible for protein-protein interaction or of mutating residues at the interface can be applied to obtain monomeric proteins.

Antigens can be classified into two categories, both of which are suitable for use with the invention. The first category is exogenous antigens, and includes all molecules found in infectious organisms. Bacterial immunogens, parasitic immunogens and viral immunogens are useful as polypeptide moieties to create multimeric or hetero-multimeric C4bp fusion proteins useful as vaccines.

Bacterial sources of these immunogens include those responsible for bacterial pneumonia, meningitis, cholera, diphtheria, pertussis, tetanus, tuberculosis and leprosy.

Parasitic sources include malarial parasites, such as Plasmodium, as well as trypanosomal and leishmania species.

Viral sources include poxviruses, e.g., smallpox virus, cowpox virus and orf virus; herpes viruses, e.g., herpes simplex virus type 1 and 2, B-virus, varicella zoster virus, cytomegalovirus, and Epstein-Barr virus; adenoviruses, e.g., mastadenovirus; papovaviruses, e.g., papillomaviruses such as HPV16, and polyomaviruses such as BK and JC virus; parvoviruses, e.g., adeno-associated virus; reoviruses, e.g., reoviruses 1, 2 and 3; orbiviruses, e.g., Colorado tick fever; rotaviruses, e.g., human rotaviruses; alphaviruses, e.g., Eastern encephalitis virus and Venezuelan encephalitis virus; rubiviruses, e.g., rubella; flaviviruses, e.g., yellow fever virus, Dengue fever viruses, Japanese encephalitis virus, Tick-borne encephalitis virus and hepatitis C virus; coronaviruses, e.g., human coronaviruses; paramyxoviruses, e.g., parainfluenza 1, 2, 3 and 4 and mumps; morbilliviruses, e.g., measles virus; pneumovirus, e.g., respiratory syncytial virus; vesiculoviruses, e.g., vesicular stomatitis virus; lyssaviruses, e.g., rabies virus; orthomyxoviruses, e.g., influenza A and B; bunyaviruses e.g., LaCrosse virus; phleboviruses, e.g., Rift Valley fever virus; nairoviruses, e.g., Congo hemorrhagic fever virus; hepadnaviridae, e.g., hepatitis B; arenaviruses, e.g., 1cm virus, Lasso virus and Junin virus; retroviruses, e.g., HTLV I, HTLV II, HIV-1 and HIV-2; enteroviruses, e.g., polio virus 1,-2 and 3, coxsackie viruses, echoviruses, human enteroviruses, hepatitis A virus, hepatitis E virus, and Norwalk-virus; rhinoviruses e.g., human rhinovirus; and filoviridae, e.g., Marburg (disease) virus and Ebola virus.

Antigens from these bacterial, viral and parasitic sources may be used in the production of multimeric proteins useful as vaccines. The multimers may comprise a mixture of monomers carrying different antigens.

Antigens from these bacterial, viral and parasitic sources can be considered as exogenous antigens because they are not normally present in the host and are not encoded in the host genome. Endogenous antigens, in contrast, are normally present in the host or are encoded in the host genome, or both. The ability to generate an immune response to an endogenous antigen is useful in treating tumours that bear that antigen, or in neutralising growth factors for the tumour. An example of the first type of endogenous antigen is HER2, the target for the monoclonal antibody called Herceptin. An example of the second, growth factor, type of endogenous antigen is gonadotrophin releasing hormone (called GnRH) which has a trophic effect on some carcinomas of the prostate gland.

Immunogens made using the present invention may be used for research or therapeutic purposes. For example, research applications include the generation of antisera to predicted gene products in genome sequence data. This requirement applies to prokaryotic, such as bacterial, and eukaryotic, including fungal and mammalian, gene products. The antigen may be any size conventional in the art for vaccines, ranging from short peptides to very large proteins.

Non-polypeptide immunogens may be, for example, carbohydrates or nucleic acids. The polysaccharide coats of Neisseria species or of Streptococcus pneumoniae species are examples of carbohydrates which may be used for the purposes of the invention.

Where a non-polypeptide immunogen is part of the product of the invention, the immunogen may be covalently attached to the first component of the product using routine synthetic methods. Generally, the immunogen may be attached to either the N- or C-terminal of a C4bp core protein comprising the first component, or to an amino acid side chain group (for example the epsilon-amino group of lysine or the thiol group of cysteine), or a combination thereof. More than one immunogen per fusion protein may be added. To facilitate the coupling, a cysteine residue may be added to the C4bp core protein, for example as the N- or C-terminus.

The present invention has many advantages in the generation of an immune response. For example, the use of multimers can permit the presentation of a number of antigens, simultaneously, to the immune system. This allows the preparation of polyvalent vaccines, capable of raising an immune response to more than one epitope, which may be present on a single organism or a number of different organisms.

Accordingly, in a further aspect the monomeric antigen may be a synthetic antigen comprising two different epitopes, either from two different organisms or from two different proteins of the same organism. An example of the latter is a fusion of a sporozoite antigen sequence, e.g. two or more NANP repeat sequences from the circumsporozoite protein joined to an MSP1 sequence. A second example of the latter is a fusion of the M2e sequence described by Neirynck et al. (Nature Medicine 5, 1157-1163, 1999) fused to a monomeric influenza hemagglutinin fragment.

Thus, vaccines formed according to the invention may be used for simultaneous vaccination against more than one disease, or to target simultaneously a plurality of epitopes on a given pathogen. The epitopes may be present in single monomer units or on different monomer units which are combined to provide a heteromultimer.

C4bp core fusion proteins in particular are useful in the context of immunisations, because the core protein is normally present in the serum or plasma of the recipient of the immunisation, and the core protein does not evoke an immune response against itself. C4bp proteins are known in a number of mammalian species, and the appropriate homologues for mammalian species may be found by those skilled in the art using standard gene cloning techniques.

### Nucleic Acids.

Products of the invention may be produced by expression of a fusion protein in a prokaryotic or eukaryotic host cell, using a nucleic acid construct encoding the protein. Where the antigen is a polypeptide, the expression of the fusion protein from a nucleic acid sequence can be used to produce a product of the invention.

Thus the invention provides a nucleic acid construct, generally DNA or RNA, which encodes a product of the invention.

The construct will generally be in the form of a replicable vector, in which sequence encoding the protein is operably linked to a promoter suitable for expression of the protein in a desired host cell.

The vectors may be provided with an origin of replication and optionally a regulator of the promoter. The vectors may contain one or more selectable marker genes. There are a wide variety of prokaryotic and eukaryotic expression vectors known as such in the art, and the present invention may utilise any vector according to the individual preferences of those of skill in the art.

A wide variety of prokaryotic host cells can be used in the method of the present invention. These hosts may include strains of Escherichia, Pseudomonas, Bacillus, Lactobacillus, Thermophilus, Salmonella, Enterobacteriacae or Streptomyces. For example, if E. coli from the genera Escherichia is used in the method of the invention, preferred strains of this bacterium to use would include derivatives of BL21(DE3) including C41(DE3), C43(DE3) or CO214(DE3), as described and made available in WO98/02559.

Even more preferably, derivatives of these strains lacking the prophage DE3 may be used when the promoter is not the T7 promoter.

Prokaryotic vectors includes vectors bacterial plasmids, e.g., plasmids derived from E. coli including ColEI, pCR1, pBR322, pMB9 and their derivatives, wider host range plasmids, e.g., RP4; phage DNAs, e.g., the numerous derivatives of phage lambda, e.g., NM989, and other DNA phages, e.g., M13 and filamentous single stranded DNA phages. These and other vectors may be manipulated using standard recombinant DNA methodology to introduce a nucleic acid of the invention operably linked to a promoter.

The promoter may be an inducible promoter. Suitable promoters include the T7 promoter, the tac promoter, the trp promoter, the lambda promoters P_{L} or P_{R} and others well known to those skilled in the art.

A wide variety of eukaryotic host cells may also be used, including for example yeast, insect and mammalian cells. Mammalian cells include CHO and mouse cells, African green monkey cells, such as COS-1, and human cells.

Many eukaryotic vectors suitable for expression of proteins are known. These vectors may be designed to be chromosomally incorporated into a eukaryotic cell genome or to be maintained extrachromosomally, or to be maintained only transiently in eukaryotic cells. The nucleic acid may be operably linked to a suitable promoter, such as a strong viral promoter including a CMV promoter, and SV40 T-antigen promoter or a retroviral LTR.

To obtain a product of the invention, host cells carrying a vector of the invention may be cultured under conditions suitable for expression of the protein, and the protein recovered from the cells of the culture medium.

### Cell Culturing.

Plasmids encoding fusion proteins in accordance with the invention may be introduced into the host cells using conventional transformation techniques, and the cells cultured under conditions to facilitate the production of the fusion protein. Where an inducible promoter is used, the cells may initially be cultured in the absence of the inducer, which may then be added once the cells are growing at a higher density in order to maximise recovery of protein.

Cell culture conditions are widely known in the art and may be used in accordance with procedures known as such.

Although WO91/11461 suggests that prokaryotic host cells may be used in the production of C4bp-based proteins, there was no experimental demonstration of such production.

Recently, it has been found that proteins fused to the C4bp core produced in the prokaryotic expression systems retain their functional activity. This is disclosed in WO2004/020639, the contents of which are incorporated herein by reference. Such methods may be used in the production of fusion proteins of the present invention.

Recovery of Protein from Culture.

Once the cells have been grown to allow for production of the protein, the protein may be recovered from the cells. Because we have found that surprisingly, the protein remains soluble, the cells will usually be spun down and lysed by sonication, for example, which keeps the protein fraction soluble and allows this fraction to remain in the supernatant following a further higher speed (e.g. 15,000 rpm for 1 hour) centrifugation.

The fusion protein in the supernatant protein fraction may be purified further by any suitable combination of standard protein chromatography techniques. We have used ion-exchange chromatography followed by gel filtration chromatography. Other chromatographic techniques, such as affinity chromatography, may also be used.

In one embodiment, we have found that heating the supernatant sample either after centrifugation of the lysate, or after any of the other purification steps will assist recovery of the protein. The sample may be heated to about 70-80° C. for a period of about 10 to 30 minutes.

Depending on the intended uses of the protein, the protein may be subjected to further purification steps, for example dialysis, or to concentration steps, for example freeze drying.

### Compositions and Uses Thereof.

Products according to the invention may be prepared in the form of a pharmaceutical composition. The product will be present with one or more pharmaceutically acceptable carriers or diluents. The composition will be prepared according to the intended use and route of administration of the product. Thus the invention provides a composition comprising a product of the invention in multimeric form together with one or more pharmaceutically acceptable carriers or diluents, and the use of such a composition in methods of immunotherapy for treatment or prophylaxis of a human or animal subject.

Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

Liquid pharmaceutically administrable compositions can, for example, be prepared by dissolving, dispersing, etc, a fusion protein of the invention with optional pharmaceutical adjuvants in a carrier, such as, for example, water, saline aqueous dextrose, glycerol, ethanol, and the like, to thereby form a solution or suspension. If desired, the composition to be administered may also auxiliary substances such as pH buffering agents and the like. Actual methods of preparing such dosage forms are known, or will be apparent, to those skilled in this art; for example, see Remington's Pharmaceutical Sciences, Mack Publishing Company, Easton, Pa., 19th Edition, 1995.

Compositions according to the invention may additionally comprise one or more adjuvants, for example mineral salts such as aluminium hydroxide or calcium phosphate, or cytokines such as IL-12 or GM-CSF. A fuller list of suitable adjuvants is given in Table 1 of Singh and O'Hagan, Nature Biotechnology, 17, 1075-1081, 1999, the disclosure of which is incorporated herein by reference.

Products according to the invention, desirably in the form of a composition or formulation may be used in methods of treatment as described herein, by administration of the product or composition thereof to a human or animal subject. The amount effective to alleviate the symptoms of the subject being treated will be determined by the physician taking into account the patient and the condition to be treated. Dosage forms or compositions containing active ingredient in the range of 0.25 to 95% with the balance made up from non-toxic carrier may be prepared.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. A more recently devised approach for parenteral administration employs the implantation of a slow-release or sustained-release system, such that a constant level of dosage is maintained. See, e.g., U.S. Pat. No. 3,710,795.

Doses of the product will be dependent upon the nature of the antigen and may be determined according to current practice for administration of that antigen in conventional vaccine formulations.

### Passive Immunisation.

In a further aspect, the invention provides a means for passive immunisation of a subject with an immune serum containing antibodies obtained by vaccination of a host subject with a product of the invention. The host subject may be a human or non-human mammal. Thus in a further aspect, the invention provides an immune serum obtained by such a method, and the use of such an immune serum in a method of treatment of the human or animal body.

### DNA Vaccines

In another aspect, the invention provides a eukaryotic expression vector comprising a nucleic acid sequence encoding a recombinant fusion protein product of the invention for use in the treatment of the human or animal body.

Such treatment would achieve its therapeutic effect by introduction of a nucleic acid sequence encoding an antigen for the purposes of raising an immune response. Delivery of nucleic acids can be achieved using a plasmid vector (in “naked” or formulated form) or a recombinant expression vector. For a review of DNA vaccination, see Ada G. and Ramshaw I, in Expert Opinion in Emerging Drugs 8, 27-35, 2003).

Various viral vectors which can be utilized for gene delivery include adenovirus, herpes virus, vaccinia or an RNA virus such as a retrovirus. The retroviral vector may be a derivative of a murine or avian retrovirus. Examples of retroviral vectors in which a single foreign gene can be inserted include, but are not limited to: Moloney murine leukaemia virus (MoMuLV), Harvey murine sarcoma virus (HaMuSV), murine mammary tumour virus (MuMTV), and Rous Sarcoma Virus (RSV). When the subject is a human, a vector such as the gibbon ape leukaemia virus (GaLV) can be utilized.

The vector will include a transcriptional regulatory sequence, particularly a promoter region sufficient to direct the initiation of RNA synthesis. Suitable eukaryotic promoters include the promoter of the mouse metallothionein I gene (Hamer et al., 1982, J. Molec. Appl. Genet. 1: 273); the TK promoter of Herpes virus (McKnight, 1982, Cell 31: 355); the SV40 early promoter (Benoist et al., 1981, Nature 290: 304); the Rous sarcoma virus promoter (Gorman et al., 1982, Proc. Natl. Acad. Sci. USA 79: 6777); and the cytomegalovirus promoter (Foecking et al., 1980, Gene 45: 101).

Administration of vectors of this aspect of the invention to a subject, either as a plasmid vector or as part of a viral vector can be affected by many different routes. Plasmid DNA can be “naked” or formulated with cationic and neutral lipids (liposomes) or microencapsulated for either direct or indirect delivery. The DNA sequences can also be contained within a viral (e.g., adenoviral, retroviral, herpesvirus, pox virus) vector, which can be used for either direct or indirect delivery. Delivery routes include but are not limited to oral, intramuscular, intradermal (Sato, Y. et al., 1996, Science 273: 352-354), intravenous, intra-arterial, intrathecal, intrahepatic, inhalation, intravaginal instillation (Bagarazzi et al., 1997, J Med. Primatol. 26:27), intrarectal, intratumour or intraperitoneal.

Thus the invention includes a vector as described herein as a pharmaceutical composition useful for allowing transfection of some cells with the DNA vector such that a therapeutic polypeptide will be expressed and have a therapeutic effect, namely to induce an immune response to an antigen. The pharmaceutical compositions according to the invention are prepared by bringing the construct according to the present invention into a form suitable for administration to a subject using solvents, carriers, delivery systems, excipients, and additives or auxiliaries. Frequently used solvents include sterile water and saline (buffered or not). One carrier includes gold particles, which are delivered biolistically (i.e., under gas pressure). Other frequently used carriers or delivery systems include cationic liposomes, cochleates and microcapsules, which may be given as a liquid solution, enclosed within a delivery capsule or incorporated into food.

An alternative formulation for the administration of gene delivery vectors involves liposomes. Liposome encapsulation provides an alternative formulation for the administration of polynucleotides and expression vectors. Liposomes are microscopic vesicles that consist of one or more lipid bilayers surrounding aqueous compartments. See, generally, Bakker-Woudenberg et al, 1993, Eur. J. Clin. Microbiol. Infect. Dis. 12 (Suppl. 1): S61, and Kim, 1993, Drugs 46: 618. Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μM to greater than 10 μM. See, for example, Machy et al., 1987, LIPOSOMES IN CELL BIOLOGY AND PHARMACOLOGY (John Libbey), and Ostro et al., 1989, American J. Hosp. Pharm. 46: 1576. Expression vectors can be encapsulated within liposomes using standard techniques. A variety of different liposome compositions and methods for synthesis are known to those of skill in the art. See, for example, U.S. Pat. No. 4,844,904, U.S. Pat. No. 5,000,959, U.S. Pat. No. 4,863,740, U.S. Pat. No. 5,589,466, U.S. Pat. No. 5,580,859, and U.S. Pat. No. 4,975,282, all of which are hereby incorporated by reference.

In general, the dosage of administered liposome-encapsulated vectors will vary depending upon such factors as the patient's age, weight, height, sex, general medical condition and previous medical history. Dose ranges for particular formulations can be determined by using a suitable animal model.

The invention is illustrated by the following examples.

### EXAMPLE 1

### Plasmodium falciparum MSP1.19-Rabbit C4bp Fusion Protein

This example illustrates the fusion of a monomeric antigen (comprising amino acids 1567-1661 of Plasmodium falciparum MSP1) to the rabbit core C4bp protein. The fusion protein, called AVD174, was expressed in, and purified from the bacterial strain C41(DE3). The fusion protein alone was used to immunise rabbits without the addition of any adjuvant.

### Cloning.

A synthetic 294 bp DNA fragment encoding residues 1567-1661 of the MSP1 protein was digested with NdeI and BamHI and ligated into pAVD181 previously digested with NdeI and BamHI. This created an open reading frame encoding the 95 amino acid MSP1.19 protein fragment fused to the C-terminal 57 residues of the alpha chain of rabbit C4bp downstream of the T7 late promoter. The construction, called pAVD174, was checked by DNA sequencing.

The nucleotide sequence encoding the AVD174 fusion protein is:

(SEQ ID NO: 21)

atgttaaacatttcccagcaccagtgcgttaagaaacagtgcccgcagaa
ctctggttgtttccgtcatctggacgagcgtgaagagtgcaaatgtctgc
tgaactacaaacaggaaggtgataaatgtgttgagaacccaaacccgacc
tgtaacgaaaacaacggcggttgtgacgctgatgctaaatgcaccgagga
agacagcggttctaacggtaagaaaatcacctgcgagtgtactaaaccgg
actcctacccgctgttcgacggtatcttttgctccGGATCCGAGGTCCCG
GAAGGCTGTGAGCAGGTGCAAGCGGGTCGCCGTCTCATGCAGTGTCTCGC
AGACCCATACGAAGTGAAAATGGCCCTGGAGGTCTACAAGCTGTCTCTGG
AGATTGAACTCCTGGAACTGCAGCGCGATAAGGCACGTAAAAGCTCTGTG
CTGCGCCAGCTGTAA

The amino acid sequence of the fusion protein AVD174 encoded by this construct is as follows:

(SEQ ID NO: 22)

MLNISQHQCV KKQCPQNSGC FRHLDEREEC KCLLNYKQEG
DKCVENPNPT CNENNGGCDA DAKCTEEDSG SNGKKITCEC
TKPDSYPLFD GIFCSGSEVP EGCEQVQAGR RLMQCLADPY
EVKMALEVYK LSLEIELLEL QRDKARKSSV LRQL

The residues 1-95 of SEQ ID NO: 22 correspond to residues 1567-1661 of Plasmodium falciparum MSP1 (the monomeric antigen), and residues 98-154 of SEQ ID NO: 22 correspond to the 57 residues of the rabbit C4bp core protein. A GS linker sequence appears between the two components.

The protein has an estimated molecular weight of 17,319 Daltons, and a theoretical pI of 5.05.

### Expression.

The plasmid pAVD174 encoding the Plasmodium falciparum-rabbit C4bp core protein was expressed in the E. coli strain C41(DE3). The transformed cells were grown in LB medium at 37° C. to an OD600 of approximately 0.6, then expression was induced with IPTG to a final concentration of 0.5 mM, and the culture was grown for a further three hours at 37° C. at which point the cells were harvested by centrifugation.

### Purification of AVD174 Fusion Protein

The protein AVD174 was purified from 1 litre of C41(DE3) cells. All of the fusion protein was found in the soluble fraction after the cells were lysed by sonication in a buffer containing 20 mM MES pH6.5 and 5 mM EDTA. The supernatant after centrifugation was loaded on a HitrapS column.

### Cationic Column (HiTrap S)

The column was equilibrated in 20 mM MES pH 6.5, 20 mM EDTA buffer (buffer A). The protein was eluted with a gradient of 10 column volumes from Buffer A to Buffer B (buffer A plus 0.5M NaCl). AVD174 eluted at a concentration of approximately 200 mM NaCl.

The HiTrapS fractions containing AVD174 were concentrated using a Millipore concentrator (cut-off 30 K) and then loaded on a Gel Filtration column.

### Gel Filtration Column (Superdex 200 26/60 Prep Grade)

A Superdex 200 26/60 column was equilibrated with 20 mM Tris buffer pH8, 150 mM NaCl, and the concentrated AVD174 protein from the HiTrapS fractions was loaded.

The protein eluted in two peaks. The correctly folded and assembled protein eluted in 156 mls, whereas an earlier, minor peak eluting at 120 mls is not correctly assembled or folded.

### Biophysical Characterisation

The oligomeric state of C4bp fusion proteins containing disulphide bonds (present in all except the murine core protein: see FIG. 1) can be checked easily by comparing the behaviour of the protein on an SDS-PAGE gel in the presence and absence of the reducing agent beta-mercaptoethanol (BME). The AVD177 protein has an apparent size of approximately 140 kDa in the absence of BME, whereas in the presence of BME, it is reduced and runs with an apparent size of just over 20 kDa.

### Mass Spectrometry

The AVD174 protein was examined by electrospray mass spectrometry after reduction by BME and alkylation by N-ethyl maleimide (NEM). Results showed the addition of 14 NEM molecules (each of 125 Da) to the protein of which the molecular weight was determined to be 19,072 Da.

### Endotoxin Levels

The level of endotoxin in the purified protein was determined using the LAL (limulus amoebocyte lysate) test kit form Biowhittaker to be 21 EU per milligram of protein.

### EXAMPLE 2

### Plasmodium falciparum MSP1.19-Human C4bp Fusion Protein

This example illustrates the fusion of a monomeric antigen (comprising amino acids 1567-1661 of Plasmodium falciparum MSP1) to the human C4bp core protein. The fusion protein was expressed in, and purified from, the bacterial strain C41(DE3). The fusion protein alone can be used to immunise humans without the addition of any adjuvant.

### Cloning.

A synthetic 294 bp DNA fragment encoding residues 1567-1661 of the MSP1 protein was digested with NdeI and BamHI and ligated into pAVD181 previously digested with NdeI and BamHI. This created an open reading frame encoding the 95 amino acid MSP1.19 protein fragment fused to the C-terminal 57 residues of the alpha chain of human C4bp downstream of the T7 late promoter. The construction, called pAVD177, was checked by DNA sequencing.

The nucleotide sequence encoding the AVD177 fusion protein is:

(SEQ ID NO: 23)

atgttaaacatttcccagcaccagtgcgttaagaaacagtgcccgcagaa
ctctggttgtttccgtcatctggacgagcgtgaagagtgcaaatgtctgc
tgaactacaaacaggaaggtgataaatgtgttgagaacccaaacccgacc
tgtaacgaaaacaacggcggttgtgacgctgatgctaaatgcaccgagga
agacagcggttctaacggtaagaaaatcacctgcgagtgtactaaaccgg
actcctacccgctgttcgacggtatcttttgctccGGATCCgagaccccc
gaaggctgtgaacaagtgctcacaggcaaaagactcatgcagtgtctccc
aaacccagaggatgtgaaaatggccctggaggtatataagctgtctctgg
aaattgaacaactggaactacagagagacagcgcaagacaatccactttg
gataaagaactataa

The amino acid sequence of the fusion protein AVD177 encoded by this construct is as follows:

(SEQ ID NO: 24)

MLNISQHQCV KKQCPQNSGC FRHLDEREEC KCLLNYKQEG
DKCVENPNPT CNENNGGCDA DAKCTEEDSG SNGKKITCEC
TKPDSYPLFD GIFCSGSETP EGCEQVLTGK RLMQCLPNPE
DVKMALEVYK LSLEIEQLEL QRDSARQSTL DKEL

The residues 1-95 of SEQ ID NO: 24 correspond to residues 1567-1661 of Plasmodium falciparum MSP1 (the monomeric antigen), and residues 98-154 of SEQ ID NO: 24 correspond to the 57 residues of the human C4bp core protein. A GS linker sequence appears between the two components.

The protein has an estimated molecular weight of 17,261 Daltons, and a theoretical pI of 4.72.

### Expression.

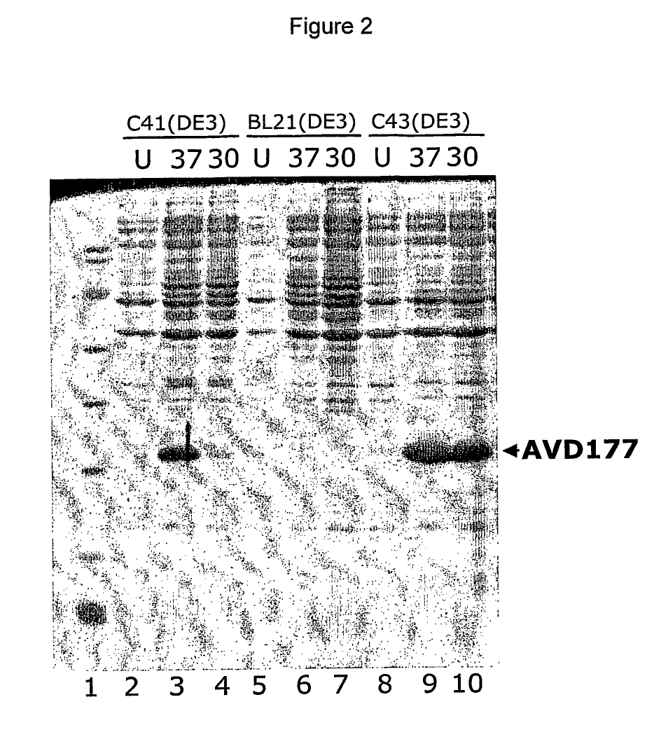

Expression of protein is shown in FIG. 2. An SDS-PAGE gel showing the expression of AVD177 protein in three different strains, C41(DE3), BL21(DE3) and C43(DE3), either uninduced (U) or after induction at 37° C. or 30° C. is presented. The lanes on the gel are as follows:

Lane 1: molecular weight markers (in descending order: 66, 60, 46, 36, 28, 20, 14, 12, 6 kDa)
Lane 2: C41(DE3) before induction
Lane 3: C41(DE3) three hours after induction at 37° C.;
Lane 4: C41(DE3) three hours after induction at 30° C.;
Lane 5: BL21(DE3) before induction
Lane 6: BL21(DE3) three hours after induction at 37° C.;
Lane 7: BL21(DE3) three hours after induction at 30° C.;
Lane 8: C43(DE3) before induction
Lane 9: C43(DE3) three hours after induction at 37° C.;
Lane 10: C43(DE3) three hours after induction at 30° C.

As can be seen, good expression was obtained in C41(DE3) and in the strain C43(DE3). In contrast, no expression was found, under the conditions tested, in the strain BL21(DE3): see FIG. 2. Cultures were grown in LB medium at 30° C. and at 37° C. to an optical density (OD600) of approximately 0.6 and then expression was induced by the addition of IPTG to a final concentration of 0.5 mM.

### Purification of AVD177 Fusion Protein

The protein AVD177 was purified from 1 litre of C41(DE3) cells grown for three hours after induction at 37° C. All of the fusion protein was found in the soluble fraction after lysis of the bacterial pellet. Cells were lysed by sonication in a buffer containing 20 mM MES pH6.5 and 5 mM EDTA. The supernatant after centrifugation was loaded on a MonoS column.

### Cationic Column (Mono S HR 10/10)

The column was equilibrated in 20 mM MES pH 6.5, 20 mM EDTA buffer (buffer A). The protein was eluted with a gradient of 10 column volumes from Buffer A to Buffer B which was buffer A plus 0.5M NaCl. AVD177 eluted at a concentration of approximately 200 mM NaCl.

The MonoS fractions containing AVD177 were concentrated using a Millipore concentrator (cut-off 30 K) and then loaded on a Gel Filtration column.

### Gel Filtration Column (Superdex 200 26/60 Prep Grade)

A Superdex 200 26/60 column was equilibrated with 20 mM Tris buffer pH8, 150 mM NaCl, and the concentrated AVD177 protein from the MonoS fractions was loaded.

The protein eluted in two peaks. The correctly folded and assembled protein eluted in 150 mls, whereas an earlier, minor peak eluting at 115 mls is not correctly assembled or folded.

### Biophysical Characterization

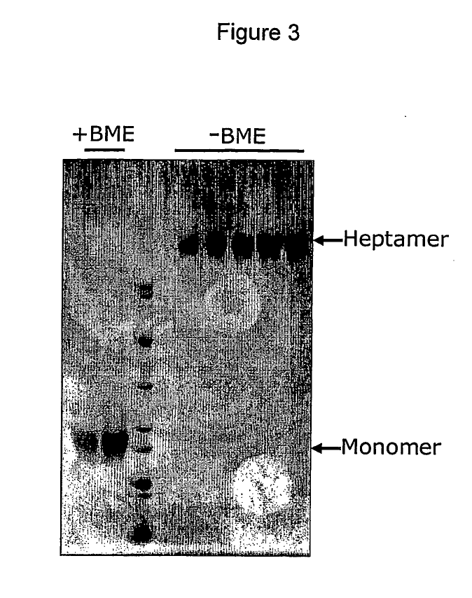

The oligomeric state of C4bp fusion proteins containing disulphide bonds (present in all except the murine core protein: see FIG. 1) can be checked easily by comparing the behaviour of the protein on an SDS-PAGE gel in the presence and absence of the reducing agent beta-mercaptoethanol (BME). The results are shown in FIG. 3, which shows an SDS-PAGE gel showing the AVD177 protein run under reducing conditions (left +BME) and under non-reducing conditions (right −BME) separated by molecular weight markers (in descending order: 66, 60, 46, 36, 28, 20, 14, 12, 6 kDa). As can be seen from FIG. 3, the AVD177 protein has an apparent size of approximately 140 kDa in the absence of BME, whereas in the presence of BME, it is reduced and runs with an apparent size of just over 20 kDa.

### Mass Spectrometry

The AVD177 protein was examined by electrospray mass spectrometry after reduction by BME and alkylation by N-ethyl maleimide (NEM). Results showed the addition of 14 NEM molecules (each of 125 Da) to the protein of which the molecular weight was determined to be 19,015 Da.

### Endotoxin Levels

The level of endotoxin in the purified protein was determined using the LAL (limulus amoebocyte lysate) test kit form Biowhittaker to be 38 EU per milligram of protein.

### EXAMPLE 3

### Mutant Plasmodium falciparum MSP1.19-Rabbit C4bp Fusion Protein

By way of example, a second Plasmodium falciparum MSP1.19-rabbit C4bp protein is described here. This differs principally in having a distinct codon usage to pAVD174 and pAVD177 for the monomeric antigen gene and also contains three amino acid changes (described in Uthaipibull et al., J Mol Biol. 307, 1381-1394, 2001). This fusion protein was called AVD178.

The nucleotide sequence encoding the AVD178 fusion protein is:

(SEQ ID NO: 25)

atgctgaatatttcccagcaccagtgcgtaaagaaacagtgtcctcagaa
ctctggttgcttccgccatctggacgaacgcgaatattgcaaatgccgtc
tgaactacaaacaggaaggtgacaagtgcgttctgaacccgaacccaact
tgtaacgagaacaacggtggctgcgatgctgatgctaaatgcactgaaga
agacagcggttctaacggcaaaaaaatcacctgcgagtgcaccaaaccgg
acagctatccgctgttcgacggcattttttgttctggatccGAGGTCCCG
GAAGGCTGTGAGCAGGTGCAAGCGGGTCGCCGTCTCATGCAGTGTCTCGC
AGACCCATACGAAGTGAAAATGGCCCTGGAGGTCTACAAGCTGTCTCTGG
AGATTGAACTCCTGGAACTGCAGCGCGATAAGGCACGTAAAAGCTCTGTG
CTGCGCCAGCTGTAA

The amino acid sequence of the fusion protein AVD178 encoded by this construct is as follows:

(SEQ ID NO: 26)

MLNISQHQCVKKQCPQNSGCFRHLDEREYCKCRLNYKQEGDKCVLNPNPT
CNENNGGCDADAKCTEEDSGSNGKKITCECTKPDSYPLFDGIFCSGSEVP
EGCEQVQAGRRLMQCLADPYEVKMALEVYKLSLEIELLELQRDKARKSSV
LRQL

The three mutant amino acids are in bold and underlined.

The residues 1-95 of SEQ ID NO: 25 correspond to residues 1567-1661 of Plasmodium falciparum MSP1 (the monomeric antigen) with three mutations, and residues 98-154 of SEQ ID NO: 24 correspond to the 57 residues of the rabbit C4bp core protein. A GS linker sequence appears between the two components.

### Expression, Purification and Characterisation of AVD178

This was carried out essentially as described for the AVD174 and AVD177 proteins. Elution from the HiTrapS column was in approximately 200 mM. On gel filtration, the oligomeric protein eluted in a volume of 159 mls. On mass spectrometry, with 14 NEM residues per monomer, the molecular mass was 19,133 Da. The endotoxin level was measured at 58EU per milligram of protein.

### EXAMPLE 4

### Plasmodium yoelii MSP1.19-Murine C4bp Fusion Protein

### Cloning

The AVD108 protein was prepared as follows: a synthetic DNA fragment encoding MSP1.19 and a part of MSP1.33 from Plasmodium yoelii was cloned as an NdeI-BamHI fragment upstream from the C-terminal 54 amino acids of the murine C4bp alpha chain.

The nucleotide sequence encoding the fusion protein AVD108 was as follows:

(SEQ ID NO: 27)

ATGAGATCTCACATTGCCTCTATTGCTTTGAACAACTTGAACAAGTCTGG
TTTGGTAGGAGAAGGTGAGTCTAAGAAGATTTTGGCTAAGATGCTGAACA
TGGACGGTATGGACTTGTTGGGTGTTGACCCTAAGCATGTTTGTGTTGAC
ACTAGAGACATTCCTAAGAACGCTGGATGTTTCAGAGACGACAACGGTAC
TGAAGAGTGGAGATGTTTGTTGGGTTACAAGAAGGGTGAGGGTAACACCT
GCGTTGAGAACAACAACCCTACTTGCGACATCAACAACGGTGGATGTGAC
CCAACCGCCTCTTGTCAAAACGCTGAATCTACCGAAAACTCCAAGAAGAT
TATTTGCACCTGTAAGGAACCAACCCCTAACGCCTACTACGAGGGTGTTT
TCTGTTCTTCTTCCGGATCCGAGGCCTCTGAAGACCTTAAGCCTGCGCTT
ACAGGCAACAAGACCATGCAGTATGTGCCAAATTCACACGATGTGAAAAT
GGCTCTGGAGATCTACAAGCTGACTCTGGAGGTTGAACTACTACAGCTCC
AGATACAAAAGGAGAAACACACTGAAGCACACTAA

The amino acid sequence of the protein AVD108 was as follows:

(SEQ ID NO: 28)

MRSHIASIAL NNLNKSGLVG EGESKKILAK MLNMDGMDLL
GVDPKHVCVD TRDIPKNAGC FRDDNGTEEW RCLLGYKKGE
GNTCVENNNP TCDINNGGCD PTASCQNAES TENSKKIICT
CKEPTPNAYY EGVFCSSSGS EASEDLKPAL TGNKTMQYVP
NSHDVKMALE IYKLTLEVEL LQLQIQKEKH TEAH

The residues 3-138 of SEQ ID NO: 28 correspond to residues 1619-1753 of Plasmodium yoelii MSP1 (the monomeric antigen), and residues 141-194 of SEQ ID NO: 28 correspond to the 54 residues of the rabbit C4bp core protein. A GS linker sequence appears between the two components, and a short restriction site encoded sequence precedes the first component.

### Expression of AVD108

The protein AVD108 was expressed in the E. coli strain C41(DE3). A three litre culture was grown in LB medium at 37° C. to an optical density (OD600) of approximately 0.6 and then expression was induced by the addition of IPTG to a final concentration of 0.7 mM. Four hours after induction, the cells were harvested by centrifugation. The cells were lysed in buffer A (50 mM Tris pH9, 5 mM EDTA) and debris removed by centrifugation.

### Purification and Characterisation of AVD108

The protein AVD108 was purified using four column chromatography steps. In the first anion-exchange chromatographic step, a DEAE HR16/10 column was used. The protein was loaded in buffer A and eluted in a gradient with buffer B, which was buffer A plus 1M NaCl. AVD108 eluted in a broad peak between 180-300 mM NaCl.

In the second hydrophobic interaction chromatographic step, the pooled fractions containing AVD108 from the DEAE column were loaded on a Macro-Prep Phenyl Sepharose column and eluted in a gradient of decreasing salt from 1M to 0M NaCl. In the final two chromatographic steps, the AVD108 protein was purified by gel filtration on a Superdex S200 26/60 column. The first time, the protein was denatured by adding urea to final concentration of 8M and incubating overnight at 4° C. The monomer eluted from this column in a volume of 203 mls. After renaturation by dialysis against Buffer H (20 mM Tris pH7.5, 150 mM NaCl) overnight at 4° C., before being loaded on the same column now equilibrated in Buffer H. The oligomers now eluted in a volume of 164 mls. By mass spectrometry, the mass was determined as 21,257 Da. Endotoxin levels by the LAL test kit from Biowhittaker was 4 EU per milligram of protein.

### EXAMPLE 5

### Immunisation Using Plasmodium falciparum MSP1.19-Rabbit C4bp Fusion Protein

### Immunisation

The AVD174 protein prepared as described above in Example 1 was used to immunise three New Zealand White (NZW) rabbits. The immunisation schedule was as follows: each rabbit received four injections at two-weekly intervals (in other words, on days 0, 14, 28 and 42). Each injection was subcutaneous and contained 345 micrograms (or 20 nanomoles) of protein in a buffered isotonic saline solution without the addition of any known adjuvant.

In parallel, three NZW rabbits were immunised with 212 micrograms (or 20 nanomoles) of AVD172 protein in Freund's adjuvant. The AVD172 is the same as AVD174 but lacks the C-terminal 57 amino acids from rabbit C4bp. It has the following amino acid sequence:

(SEQ ID NO: 29)

MLNISQHQCV KKQCPQNSGC FRHLDEREEC KCLLNYKQEG
DKCVENPNPT CNENNGGCDA DAKCTEEDSG SNGKKITCEC
TKPDSYPLFD GIFCS.

The immunisation schedule was as follows: each rabbit received four injections at two-weekly intervals (in other words, on days 0, 14, 28 and 42). The first injection for each rabbit was in Complete Freund's Adjuvant and was administered intradermally, whereas the three following injections were given in Incomplete Freund's Adjuvant, and were administered subcutaneously.

### Antibody Titres

Antibody titres one week after the last injection (i.e; on day 63) against MSP1 on the surface of Plasmodium falciparum merozoites were measured by indirect immunofluorescence (as described in Ling et al. Vaccine 15, 1562-1567, 1997 for P. yoelii) on acetone-fixed smears of P. falciparum infected erythrocytes.

The two highest titres in the rabbits immunised with the AVD174 protein were 1/81,920. In the rabbits immunised with the AVD172 protein in Freund's adjuvant, the two highest titres were 1/20,480.

This demonstrated that fusing a monomeric antigen to a C4bp core can induce higher antibody titres than administering the same monomeric antigen in Freund's adjuvant.

### Characterisation of Antibodies Produced

High titres of antibodies may not suffice to prevent or treat an infection, and it is known that the specificity of the antibodies produced on immunisation with an antigen can be of critical importance. Guevara Patino et al. (J. Exp. Med. 186, 1689-1699, 1997) have described in detail methods for assaying blocking and inhibitory antibodies against MSP1.19. These methods were used to see if there were inhibitory antibodies present (which are useful because they block the processing of MSP1.42 into MSP1.33 and MSP1.19 and thus prevent erythrocyte invasion).

Inhibitory antibodies were only found among the antibodies induced by the AVD174 protein. None were found in the antisera of rabbits immunised with AVD172 protein. In contrast, AVD172 in Freund's adjuvant can induce blocking antibodies, as can natural Plasmodium infections in man and these are deleterious (see Guevara Patino et al., op. cit.).

### EXAMPLE 6

### Immunisation Using Plasmodium yoelii MSP1.19-Murine C4bp Fusion Protein

### Immunisation of Mice

The AVD108 protein prepared as described in Example 5 was used to immunise six BALB/c mice. No adjuvant was used, and the protein was in a buffered isotonic saline solution. Forty micrograms (1.9 nanomoles) of protein was used per injection. Each mouse was injected three times, subcutaneously, at four-weekly intervals (in other words, on days 0, 28 and 56). In parallel, six BALB/c mice were immunised with twenty-three micrograms (also 1.9 nanomoles) of the AVD183 protein, which is the same as AVD108 but lacking the murine C4bp C-terminal 54 amino acids (i.e. it is the P. yoelii MSP1.19 protein alone). Twenty-three micrograms of this protein (in the same buffered isotonic saline solution used for AVD108) was used per injection. Each mouse was injected three times, subcutaneously, on days 0, 28 and 56.

### Antibody Titres

Antibody titres two weeks after the last injection (i.e; on day 70) against MSP1 on the surface of Plasmodium yoelii merozoites were measured by indirect immunofluorescence (as described in Ling et al. Vaccine 15, 1562-1567, 1997) on acetone-fixed smears of P. yoelii infected erythrocytes.

The results showed that five of six mice immunised with the AVD108 protein had titres of 1/40,960, while the sixth mouse had a titre of 1/10,240. In contrast, no antibodies against MSP1 could be detected in any of the mice immunised with the AVD183 protein at a dilution of 1/80. This demonstrated that fusing the monomeric MSP1.19 antigen to a C4bp core could increase the titre of antibodies obtained up to five-hundred-fold.

### Parasite Challenge

Both groups of six mice immunised as described above were challenged with a lethal dose of 5,000 P. yoelii infected erythrocytes. Again, this assay has been described by Ling et al. (op. cit.). The six mice immunised with the AVD183 protein all died within seven days of the parasite challenge. On the other hand, five of the six mice immunised with AVD108 were alive and free of parasites in their blood (as assessed by Giemsa staining of thin blood smears examined microscopically). The sixth mouse, which had a day 70 titre of 1/10,240 died nineteen days after the challenge; over 70% of this mouse's erythrocytes were seen to be infected by Giemsa staining on day 19.

In conclusion, this challenge experiment demonstrated that immunisation with a monomeric antigen fused to a C4bp core protein alone in the absence of any known adjuvants could protect against an otherwise lethal Plasmodium infection. It is believed at present that this represents the first instance of successful vaccination against Plasmodium infection using just a single protein unaccompanied by any known adjuvant.

### EXAMPLE 7

### Influenza Hemagglutinin-C4bp Fusion Proteins

This example illustrates the fusion of a monomeric antigen (comprising residues 91-261 of the HA1 hemagglutinin protein of influenza A virus) to the human, rabbit and murine core C4bp proteins. Normally, the full-length HA1 is assembled into a trimer on the surface of virions cells, so that using only this peptide fragment effectively converts it into a monomeric antigen. The fusion proteins, called AVD272 to AVD274, are expressed in, and purified from the bacterial strain C41(DE3). These fusion proteins alone are used to immunise mice and rabbits without the addition of any adjuvant.

In AVD272, the HA1 fragment (described in Jeon and Arnon, Viral Immunology, 15, 165-176, 2002) is fused to the murine C4bp scaffold.

The amino acid sequence of AVD272 is as follows:

(SEQ ID NO: 30)

kafsncypyd vpdyaslrsl vassgtlefi tegftwtgvt
qnggsnackr gpgsgffsrl nwltksgsty pvlnvtmpnn
dnfdklyiwg ihhpstnqeq tslyvqasgr vtvstrrsqq
tiipnigsrp wvrglssris iywtivkpgd vlvinsngnl
iaprgyfkmr GSEASEDLKP ALTGNKTMQY VPNSHDVKMA
LEIYKLTLEV ELLQLQIQKE KHTEAH

In AVD273, the HA1 fragment is fused to the rabbit C4bp scaffold.

The amino acid sequence of AVD273 is as follows:

(SEQ ID NO: 31)

kafsncypyd vpdyaslrsl vassgtlefi tegftwtgvt
qnggsnackr gpgsgffsrl nwltksgsty pvlnvtmpnn
dnfdklyiwg ihhpstnqeq tslyvqasgr vtvstrrsqq
tiipnigsrp wvrglssris iywtivkpgd vlvinsngnl
iaprgyfkmr GSEVPEGCEQ VQAGRRLMQC LADPYEVKMA
LEVYKLSLEI ELLELQRDKA RKSSVLRQL

In AVD274, the HA1 fragment was fused to the human C4bp scaffold.

The amino acid sequence of AVD274 is as follows:

(SEQ ID NO: 32)

kafsncypyd vpdyaslrsl vassgtlefi tegftwtgvt
qnggsnackr gpgsgffsrl nwltksgsty pvlnvtmpnn
dnfdklyiwg ihhpstnqeq tslyvqasgr vtvstrrsqq
tiipnigsrp wvrglssris iywtivkpgd vlvinsngnl
iaprgyfkmr GSETPEGCEQ VLTGKRLMQC LPNPEDVKMA
LEVYKLSLEI EQLELQRDSA RQSTLDKEL

Mice are immunised three times subcutaneously with the AVD272 protein (2 nanomoles) without the addition of any adjuvant at two-weekly intervals (i.e. on days 0, 14, and 28) and show appreciable antibody titres (as determined essentially as described by Jeon et al., op.cit.) and significant protection against a lethal challenge with 5 LD50 doses of the mouse adapted A/PR/8/34 strain.

### EXAMPLE 8

### Influenza M2 Peptide-C4bp Fusion Proteins

This example illustrates the fusion of a monomeric antigen (comprising residues 2-24, the extracellular part, of the M2 protein of influenza A virus) to the human, rabbit and murine core C4bp proteins. Normally, the full-length M2 is assembled into a tetramer in virions and infected cells, so that using only this peptide fragment effectively converts it into a monomeric antigen. The fusion proteins, called AVD275 to AVD278, were expressed in, and purified from the bacterial strain C41(DE3). These fusion proteins alone were used to immunise mice and rabbits without the addition of any adjuvant.

In AVD275, the extracellular M2 peptide (described in Neirynck et al. Nature Medicine 5, 1157-1163, 1999) was fused to the murine C4bp scaffold.

The amino acid sequence of AVD275 was as follows:

(SEQ ID NO: 33)

SLLTEVETPI RNEWGCRCND SSDGSEASED LKPALTGNKT
MQYVPNSHDV KMALEIYKLT LEVELLQLQI QKEKHTEAH

In AVD276, the extracellular M2 peptide was fused to the rabbit C4bp scaffold.

The amino acid sequence of AVD276 was as follows:

(SEQ ID NO: 34)

SLLTEVETPI RNEWGCRCND SSDGSEVPEG CEQVQAGRRL
MQCLADPYEV KMALEVYKLS LEIELLELQR DKARKSSVLR
QL

In AVD277, the extracellular M2 peptide was fused to the human C4bp scaffold.

The amino acid sequence of AVD277 was as follows:

(SEQ ID NO: 35)

SLLTEVETPI RNEWGCRCND SSDGSETPEG CEQVLTGKRL
MQCLPNPEDV KMALEVYKLS LEIEQLELQR DSARQSTLDK
EL

In AVD278, a variant of the extracellular M2 peptide in which both cysteines were replaced by serine residues was fused to the human C4bp scaffold.

The amino acid sequence of AVD278 was as follows:

(SEQ ID NO: 36)

SLLTEVETPI RNEWGSRSND SSDGSETPEG CEQVLTGKRL
MQCLPNPEDV KMALEVYKLS LEIEQLELQR DSARQSTLDK
EL

Mice immunised three times subcutaneously with the AVD275 protein (2 nanomoles) without the addition of any adjuvant at two-weekly intervals (i.e. on days 0, 14, and 28) show appreciable antibody titres (as determined essentially as described by Neirynck et al., op.cit.) and significant protection against a lethal challenge with 5 LD50 doses of the mouse adapted A/PR/8/34 strain.

1. A product which comprises:
a C4bp core protein; and
a monomeric antigen.

2. A product according to claim 1 wherein said the C4bp core consists of the residues 1-57 of SEQ ID NO:1 or the corresponding residues of homologue thereof, or a fragment of at least 47 amino acids of SEQ ID NO:1 or homologue thereof.

3. A product according to claim 2 wherein said homologue is any one of SEQ ID NO:2 to 20.

4. A product according to claim 2 wherein said homologue is a variant of any one of SEQ ID NO:1 to 20 having at least 70% amino acid identity thereto.

5. A product according to claim 1 wherein said monomeric antigen is fused to the N- or C-terminal of said C4bp core protein.

6. A product according to claim 5 wherein said fusion is via a flexible linker.

7. A product according to claim 1 wherein said monomeric antigen is a monomeric antigenic fragment of a Plasmodium merozoite surface protein 1.

8. A product according to claim 1 wherein said monomeric antigen is a monomeric antigenic fragment of influenza virus hemagglutinin protein or the influenza M2e peptide.

9. A composition comprising the product of claim 1 together with a pharmaceutically acceptable diluent, carrier or adjuvant.

10. A product according to claim 1 for use in a method of treatment of the human or animal body.

11. A method of immunotherapy of malaria, comprising administering to an individual an effective amount of a product according to claim 7.

12. The method of claim 11, wherein said individual is infected with a malarial parasite.

13. The method of claim 11 for preventative vaccination.

14. A product according to claim 7 for the treatment or prevention of malaria.

15. (canceled)

16. A method of producing antibodies against a Plasmodium parasite, said method comprising introducing a product according to claim 7 into a non-human mammal, and recovering immune serum from said mammal.

17. A method of passive immunisation against a disease of a subject, said method comprising administering to said subject an immune serum containing antibodies obtained by vaccination of a host subject with a product according to claim 1.

18. A method of passive immunotherapy of malaria in a human subject, said method comprising administering to said human an effective amount of an immune serum produced according to claim 16.

19. The immune serum obtained by the method of claim 16 for use in a method of immunotherapy of malaria in a human subject.

20. A method of immunotherapy of influenza, comprising administering to an individual an effective amount of a product according to claim 8.

21. The method of claim 20, wherein said individual is infected with influenza virus.

22. The method of claim 20 for preventative vaccination.

23. A product according to claim 8 for the treatment or prevention of influenza.

24. (canceled)

25. A method of producing antibodies against influenza, said method comprising introducing a product according to claim 8 into a non-human mammal, and recovering immune serum from said mammal.

26. A method of passive immunotherapy of influenza in a human subject, said method comprising administering to said human an effective amount of an immune serum produced according to claim 25.

27. The immune serum obtained by the method of claim 25 for use in a method of immunotherapy of influenza in a human subject.

28. A method of making a product comprising:
a C4bp core protein; and
a non-polypeptide monomeric antigen,
the method comprising expressing nucleic acid encoding the first component, joining said fusion protein to the second component, and recovering the product.

29. A method of making a product comprising a fusion of:
a C4bp core protein; and
a polypeptide monomeric antigen,
the method comprising expressing nucleic acid encoding the fusion, and recovering the product.

30. The method of claim 28 wherein the nucleic acid is expressed in a prokaryotic host cell.

31. A method according to claim 30 wherein the fusion protein is recovered in multimeric form.

32. A method for increasing the immunogenicity of a monomeric antigen, said method comprising joining said antigen to a C4bp core protein.

33. An expression vector comprising a nucleic acid sequence encoding a fusion protein of
a C4bp core protein; and
a polypeptide monomeric antigen,
operably linked to a promoter functional in a host cell.

34. The expression vector of claim 33 wherein said C4bp core protein is as defined above.

35. The expression vector of claim 33 wherein said monomeric antigen is as defined above.

36. A bacterial host cell transformed with the expression vector of claim 33.

37. A eukaryotic host cell transformed with the vector of claim 33.

38. (canceled)


SEQUENCE LISTING (44 sequences)

SEQ ID NO: 1 - protein, 57 aa, Homo sapiens
ETPEGCEQVLTGKRLMQCLPNPEDVKMALEVYKLSLEIEQLELQRDSARQSTLDKEL

SEQ ID NO: 2 - protein, 57 aa, Pan sp.
ETPEGCEQVLTGKRLMQCLPNPEDVKMALEVYKLSLEIEQLELQRDSARQYTLDKEL

SEQ ID NO: 3 - protein, 57 aa, Macaca mulatta
ETPEGCEQVLAGKRLMQCLPNPEDVKMALEVYKLSLEIEQLELQRDRARQSTLDKEL

SEQ ID NO: 4 - protein, 57 aa, Macaca fascicularis
ETPEGCEQVLAGKRLMQCLPNPEDVKMALEVYKLSLEIEQLELQRDRARQSTWDKEL

SEQ ID NO: 5 - protein, 57 aa, Oryctolagus cuniculus
EVPEGCEQVQAGRRLMQCLADPYEVKMALEVYKLSLEIELLELQRDKARKSSVLRQL

SEQ ID NO: 6 - protein, 57 aa, Canis familiaris
VVPEGCEHILKGRKTMQCLPNPEDVKMALEIYKLSLDIELLELQRDRAKESTVQSPV

SEQ ID NO: 7 - protein, 55 aa, Rattus sp.
EVPKDCEHVFAGKKLMQCLPNSNDVKMALEVYKLTLEIKQLQLQIDKAKHVDREL

SEQ ID NO: 8 - protein, 57 aa, Sus scrofa
EYPEDCEQVHEGKKLMQCLPNLEEIKLALELYKLSLETKLLELQIDKEKKAKAKYSI

SEQ ID NO: 9 - protein, 57 aa, Sus scrofa
EYPEDCEQVHEGKKLMECLPTLEEIKLALALYKLSLETNLLELQIDKEKKAKAKYST

SEQ ID NO: 10 - protein, 57 aa, Equus caballus
EIAEGCEQVLAGRKIMQCLPKPEDVRTALELYKLSLEIKQLEKKLEKEEKCTPEVQE

SEQ ID NO: 11 - protein, 67 aa, Bos sp.
EYPEGCEQVVTGRKLLQCLSRPEEVKLALEVYKLSLEIEILQTNKLKKEAFLLREREKNVTCDFNPE

SEQ ID NO: 12 - protein, 52 aa, Bos sp.
EYPEGCEQVVTGRKLLKCLSRPEEVKLALEVYKLSLEIALLELQIDKPKDAS

SEQ ID NO: 13 - protein, 52 aa, Cavia porcellus
EVPENCEQVIVGKKLMKCLSNPDEAQMALQLYKLSLEAELLRLQIVKARQGS

SEQ ID NO: 14 - protein, 54 aa, Mus sp.
EASEDLKPALTGNKTMQYVPNSHDVKMALEIYKLTLEVELLQLQIQKEKHTEAH

SEQ ID NO: 15 - protein, 51 aa, Bos sp.
VSAEVCEAVFKGQKLLKCLPNAMEVKMALEVYKLSLEIEKLEQEKRKLEIA

SEQ ID NO: 16 - protein, 66 aa, Cavia porcellus
EVPEECKQVAAGRKLLECLPNPSDVKMALEVYKLSLEIEQLEKEKYVKIQEKFSKKEMKQLTSALH

SEQ ID NO: 17 - protein, 59 aa, Mus sp.
EVLEDCRIVSRGAQLLHCLSSPEDVHRALKVYKLFLEIERLEHQKEKWIQLHRKPQSMK

SEQ ID NO: 18 - protein, 63 aa, Rattus sp.
EGPEDCEIVNKGRQLLQCLSSPEDVQRALEVYKLSLEIERLEQQREKRTSVHRKAHYTKVDGP

SEQ ID NO: 19 - protein, 67 aa, Homo sapiens
EAPEGCEQVLTGRKLMQCLPSPEDVKVALEVYKLSLEIKQLEKERDKLMNTHQKFSEKEEMKDLFFP

SEQ ID NO: 20 - protein, 52 aa, Artificial sequence
  Consensus
EVPEGCEQVLTGKKLMQCLPNPEDVKMALEVYKLSLEIELLELQIDKARQGS

SEQ ID NO: 21 - DNA, 465 bp, Artificial sequence
  Sequence encoding the AVD174 fusion protein
atgttaaacatttcccagcaccagtgcgttaagaaacagtgcccgcagaactctggttgtttccgtcatctggacgagcgtgaagagtgcaaatgtctgctgaactacaaacaggaaggtgataaatgtgttgagaacccaaacccgacctgtaacgaaaacaacggcggttgtgacgctgatgctaaatgcaccgaggaagacagcggttctaacggtaagaaaatcacctgcgagtgtactaaaccggactcctacccgctgttcgacggtatcttttgctccggatccgaggtcccggaaggctgtgagcaggtgcaagcgggtcgccgtctcatgcagtgtctcgcagacccatacgaagtgaaaatggccctggaggtctacaagctgtctctggagattgaactcctggaactgcagcgcgataaggcacgtaaaagctctgtgctgcgccagctgtaa

SEQ ID NO: 22 - protein, 154 aa, Artificial sequence
  AVD174 fusion protein
MLNISQHQCVKKQCPQNSGCFRHLDEREECKCLLNYKQEGDKCVENPNPTCNENNGGCDADAKCTEEDSGSNGKKITCECTKPDSYPLFDGIFCSGSEVPEGCEQVQAGRRLMQCLADPYEVKMALEVYKLSLEIELLELQRDKARKSSVLRQL

SEQ ID NO: 23 - DNA, 465 bp, Artificial sequence
  The nucleotide sequence encoding the AVD177 fusion protein
atgttaaacatttcccagcaccagtgcgttaagaaacagtgcccgcagaactctggttgtttccgtcatctggacgagcgtgaagagtgcaaatgtctgctgaactacaaacaggaaggtgataaatgtgttgagaacccaaacccgacctgtaacgaaaacaacggcggttgtgacgctgatgctaaatgcaccgaggaagacagcggttctaacggtaagaaaatcacctgcgagtgtactaaaccggactcctacccgctgttcgacggtatcttttgctccggatccgagacccccgaaggctgtgaacaagtgctcacaggcaaaagactcatgcagtgtctcccaaacccagaggatgtgaaaatggccctggaggtatataagctgtctctggaaattgaacaactggaactacagagagacagcgcaagacaatccactttggataaagaactataa

SEQ ID NO: 24 - protein, 154 aa, Artificial sequence [sequence omitted]

SEQ ID NO: 25 - DNA, 465 bp, Artificial sequence
  The nucleotide sequence encoding the AVD178 fusion protein.
atgctgaatatttcccagcaccagtgcgtaaagaaacagtgtcctcagaactctggttgcttccgccatctggacgaacgcgaatattgcaaatgccgtctgaactacaaacaggaaggtgacaagtgcgttctgaacccgaacccaacttgtaacgagaacaacggtggctgcgatgctgatgctaaatgcactgaagaagacagcggttctaacggcaaaaaaatcacctgcgagtgcaccaaaccggacagctatccgctgttcgacggcattttttgttctggatccgaggtcccggaaggctgtgagcaggtgcaagcgggtcgccgtctcatgcagtgtctcgcagacccatacgaagtgaaaatggccctggaggtctacaagctgtctctggagattgaactcctggaactgcagcgcgataaggcacgtaaaagctctgtgctgcgccagctgtaa

SEQ ID NO: 26 - protein, 154 aa, Artificial sequence
  The fusion protein AVD178
MLNISQHQCVKKQCPQNSGCFRHLDEREYCKCRLNYKQEGDKCVLNPNPTCNENNGGCDADAKCTEEDSGSNGKKITCECTKPDSYPLFDGIFCSGSEVPEGCEQVQAGRRLMQCLADPYEVKMALEVYKLSLEIELLELQRDKARKSSVLRQL

SEQ ID NO: 27 - DNA, 585 bp, Artificial sequence
  The nucleotide sequence encoding the fusion protein AVD108.
atgagatctcacattgcctctattgctttgaacaacttgaacaagtctggtttggtaggagaaggtgagtctaagaagattttggctaagatgctgaacatggacggtatggacttgttgggtgttgaccctaagcatgtttgtgttgacactagagacattcctaagaacgctggatgtttcagagacgacaacggtactgaagagtggagatgtttgttgggttacaagaagggtgagggtaacacctgcgttgagaacaacaaccctacttgcgacatcaacaacggtggatgtgacccaaccgcctcttgtcaaaacgctgaatctaccgaaaactccaagaagattatttgcacctgtaaggaaccaacccctaacgcctactacgagggtgttttctgttcttcttccggatccgaggcctctgaagaccttaagcctgcgcttacaggcaacaagaccatgcagtatgtgccaaattcacacgatgtgaaaatggctctggagatctacaagctgactctggaggttgaactactacagctccagatacaaaaggagaaacacactgaagcacactaa

SEQ ID NO: 28 - protein, 194 aa, Artificial sequence
  Protein AVD108
MRSHIASIALNNLNKSGLVGEGESKKILAKMLNMDGMDLLGVDPKHVCVDTRDIPKNAGCFRDDNGTEEWRCLLGYKKGEGNTCVENNNPTCDINNGGCDPTASCQNAESTENSKKIICTCKEPTPNAYYEGVFCSSSGSEASEDLKPALTGNKTMQYVPNSHDVKMALEIYKLTLEVELLQLQIQKEKHTEAH

SEQ ID NO: 29 - protein, 95 aa, Artificial sequence
  AVD172
MLNISQHQCVKKQCPQNSGCFRHLDEREECKCLLNYKQEGDKCVENPNPTCNENNGGCDADAKCTEEDSGSNGKKITCECTKPDSYPLFDGIFCS

SEQ ID NO: 30 - protein, 226 aa, Artificial sequence
  AVD272
KAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRGSEASEDLKPALTGNKTMQYVPNSHDVKMALEIYKLTLEVELLQLQIQKEKHTEAH

SEQ ID NO: 31 - protein, 229 aa, Artificial sequence
  AVD273
KAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRGSEVPEGCEQVQAGRRLMQCLADPYEVKMALEVYKLSLEIELLELQRDKARKSSVLRQL

SEQ ID NO: 32 - protein, 229 aa, Artificial sequence
  AVD274
KAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRGSETPEGCEQVLTGKRLMQCLPNPEDVKMALEVYKLSLEIEQLELQRDSARQSTLDKEL

SEQ ID NO: 33 - protein, 79 aa, Artificial sequence
  AVD275
SLLTEVETPIRNEWGCRCNDSSDGSEASEDLKPALTGNKTMQYVPNSHDVKMALEIYKLTLEVELLQLQIQKEKHTEAH

SEQ ID NO: 34 - protein, 82 aa, Artificial sequence
  AVD276
SLLTEVETPIRNEWGCRCNDSSDGSEVPEGCEQVQAGRRLMQCLADPYEVKMALEVYKLSLEIELLELQRDKARKSSVLRQL

SEQ ID NO: 35 - protein, 82 aa, Artificial sequence
  AVD277
SLLTEVETPIRNEWGCRCNDSSDGSETPEGCEQVLTGKRLMQCLPNPEDVKMALEVYKLSLEIEQLELQRDSARQSTLDKEL

SEQ ID NO: 36 - protein, 82 aa, Artificial sequence [sequence omitted]

SEQ ID NO: 37 - protein, 47 aa, Artificial sequence
  Example of fragment or variant of the C4bp core protein
GCEQVLTGKRLMQCLPNPEDVKMALEVYKLSLEIEQLELQRDSARQS

SEQ ID NO: 38 - protein, 51 aa, Artificial sequence
  Example of fragment or variant of the C4bp core protein
ETPEGCEQVLTGKRLMQCLPNPEDVKMALEVYKLSLEIEQLELQRDSARQS

SEQ ID NO: 39 - protein, 53 aa, Artificial sequence
  Example of fragment or variant of the C4bp core protein
GSEQVLTGKRLMQSLPNPEDVKMALEVYKLSLEIEQLELQRDSARQSTLDKEL

SEQ ID NO: 40 - protein, 57 aa, Artificial sequence
  Example of fragment or variant of the C4bp core protein
ETPEGCEQVLTGKRLMQCLPNPEDVKMALEIYKLTLEIEQLELQRDSARQSTLDKEL

SEQ ID NO: 41 - protein, 57 aa, Artificial sequence
  Example of fragment or variant of the C4bp core protein
ETPEGCEQVLTGKRLMQCLPNPEDVKMALEIYKLSLEIKQLELQRDSARQSTLDKEL

SEQ ID NO: 42 - protein, 50 aa, Artificial sequence
  Example of fragment or variant of the C4bp core protein
EGCEQILTGKRLMQCLPDPEDVKMALEIYKLSLEIKQLELQRDRARQSTL

SEQ ID NO: 43 - protein, 57 aa, Artificial sequence
  Example of fragment or variant of the C4bp core protein
ETPEGCEQVLTGKRLMQCLPNPEDVKMALEVYKLSLEIKQLELQRDRARQSTLDKEL

SEQ ID NO: 44 - protein, 52 aa, Artificial sequence
  Example of fragment or variant of the C4bp core protein
EGCEQILTGKRLMQCLPNPEDVKMALEIYKLSLEIEQLELQRDRARQSTLDK